(19) United States
(12) Patent Application Publication
(10) Pub. No.: US 2004/0213839 A1
(43) Pub. Date: Oct. 28, 2004

(54) Methods and compositions for reducing the taste of pharmaceutically active agents

(76) Inventors: Andrew Favara, Favara, NJ (US); David F. Erkoboni, Pennington, NJ (US)

Correspondence Address: Donald O Nickey, Cardinal Health Inc, 7000 Cardinal Place, Dublin, OH, 43017, US

(21) Appl. No.: 10/467,402

(22) PCT Filed: Feb. 4, 2002
(86) PCT No.: PCT/US02/03199

Related U.S. Application Data
(60) Provisional application No. 60/266,721, filed on Feb. 5, 2001.

(51) Int. Cl. A61K 9/20; D04H 1/00

(52) U.S. Cl. 424/464; 264/109

(57) ABSTRACT

The present invention provides methods and compositions for reducing the perception of poor-tasting pharmaceutically active agents in the oral cavity. The invention provides particles containing one or more pharmaceutically active agents, flavorants and cellulosic materials, as well as methods for making same and for incorporating same into pharmaceutical dosage forms. In certain embodiments, the particles have a diameter of up to about 1000 micrometers and include the pharmaceutically active agent, a flavorant, and at least one cellulosic material that is microcrystalline cellulose, microcrystalline cellulose coprocessed with a hydrocolloid, or any combination thereof, individually or in admixture with a hydrocolloid.

### FIELD OF THE INVENTION

The present invention relates to pharmaceutical compositions in which an unpleasant taste associated with a pharmaceutically active agent is reduced. The invention also relates to methods for preparing pharmaceutical dosage forms in which flavorants are combined with active agents.

### BACKGROUND OF THE INVENTION

It is known that many widely used pharmaceutically active agents, such as ibuprofen, leave an unpleasant taste in the mouth of someone who ingests a chewable dosage form containing the active agent. Flavorants such as vanilla, chocolate, anise, fruit flavors and the like have been proposed for use with and are used with unpleasant-tasting pharmaceutically active agents such as ibuprofen. Such flavorants, however, have not proven to be reliable agents for reducing the taste of such agents. The most commonly used methods for improving unpleasant-tasting pharmaceutically active agents typically have involved coating drug-containing particles with a barrier coating that will not dissolve in the mouth but will readily dissolve in gastric fluids. However, the coating can break when chewed, allowing the pharmaceutically active agent to be released. Coatings that resist breaking when chewed tend to inhibit bioavailability and/or release of the agent.

U.S. Pat. No. 4,835,187, in the name of Reuter, et al., discloses a therapeutic, taste-neutral form of spray dried ibuprofen powder consisting essentially of 40% to 70% by weight ibuprofen, 15% to 50% by weight of a cellulose material selected from ethylcellulose, hydroxyethylcellulose, hydroxypropylmethylcellulose and admixtures thereof and 5% to 40% by weight colloidal silica. The powder is obtained by spray drying a suspension of the colloidal silica in a lower alkanol solution of the ibuprofen and the cellulose material. The patent discloses mixing two separate slurries of ingredients, filtering them, then mixing the two filtrates and spray drying the combined slurry.

U.S. Pat. No. 5,215,755, in the name of Roche, et al., describes chewable tablets and taste masked granules for making the same. The granules are prepared by rotogranulation of the active with polyvinylpyrrolidone, sodium starch glycolate and sodium lauryl sulfate, and are coated with hydroxyethyl cellulose or a mixture of hydroxyethyl cellulose and hydroxypropylmethyl cellulose. The coating is said to achieve a beneficial balance of taste masking and bioavailability. Microcrystalline cellulose is disclosed as a binder for the granules in the compressed chewable tablets.

More effective methods for neutralizing unpleasant flavors associated with pharmaceutically active agents are desired.

### SUMMARY OF THE INVENTION

In one aspect, the present invention provides particles containing one or more pharmaceutically active agents. In preferred embodiments, the particles include the pharmaceutically active agent, a flavorant, and at least one cellulosic material that is microcrystalline cellulose, microcrystalline cellulose coprocessed with a hydrocolloid, or any combination thereof, individually or in admixture with a hydrocolloid.

The present invention also provides methods for preparing such particles. One preferred method comprises forming the particles from a wet granulation that includes a solvent portion that includes water and/or some other suitable solvent and a non-solvent portion that includes a pharmaceutically active agent, a flavorant, and a cellulosic material that is microcrystalline cellulose, microcrystalline cellulose coprocessed with a hydrocolloid, or any combination thereof, individually or in admixture with a hydrocolloid.

In a further aspect, the present invention provides tablets that are prepared by compressing the particles of the invention together, preferably with one or more pharmaceutically acceptable excipients and/or adjuvants.

### DETAILED DESCRIPTION OF THE INVENTION

It has been discovered in connection with the present invention that an unpleasant or objectionable taste associated with a pharmaceutically active agent can be effectively reduced by maintaining the agent, a flavorant, and a cellulosic material in relatively close physical contact with one another in an oral dosage form. Although not wishing to be bound by any particular theory, it is believed that such relatively close physical contact facilitates dissolution of the pharmaceutically active agent and flavorant at relatively similar rates, thereby maximizing the extent to which the pharmaceutically active agent and flavorant are present together at any relevant point in time within the oral cavity.

It is believed that many prior approaches to reducing the taste of the pharmaceutical active agents met with limited success because they failed to achieve adequate association of the pharmaceutically active agent and flavorant within a dosage form for the period of time the dosage resides in the oral cavity. Flavorants in the resulting dosage forms typically dissolved at too rapid a rate relative to the pharmaceutically active agent and did not reside within the oral cavity for as long a period as the pharmaceutically active agent. This resulted in less than effective taste reduction. It is believed that by more closely matching the respective dissolution profiles of the active agent and the flavorant (such as by placing them in relatively close physical contact and/or modifying their respective solubility, particle size, surface area and/or morphology), the flavorant can be used to more optimally reduce the taste of the active agent.

The methods of the invention involve mixing a pharmaceutically active agent with a flavorant and a cellulosic material to form a relatively free-flowing composition. (As will be recognized, one or more of each of the foregoing ingredients can be employed. Thus, the article “a” is not intended in this context to be limiting.). Representative of such compositions are those that are prepared by mixing about 40 to about 95 weight percent of the pharmaceutically active agent, about 0.01 to about 25 weight percent of the flavorant, and about 1 to about 60 weight percent of the cellulosic material. Preferred compositions are those prepared by mixing about 60 to about 95 weight percent of the pharmaceutically active agent, about 0.01 to about 15 weight percent of the flavorant, and about 1 to about 40 weight percent of the cellulosic material A particularly preferred class of compositions is prepared by mixing about 70 to about 95 weight percent of the pharmaceutically active agent, about 0.01 to about 10 weight percent of the flavorant, and about 1 to about 30 weight percent of the cellulosic material.

Virtually any pharmaceutically active agent can be used. Preferred agents are those that can be processed into free-flowing powders. Representative pharmaceutically active agents include: analgesics such as acetaminophen, ibuprofen, ketoprofen, indomethacin, naproxen; antibiotics such as erythromycin, cephalosporin and minocycline HCl; cough and cold agents such as dextromethorphan hydrobromide, ephedrine sulfate, guaifenesin, promethazine hydrochloride, and pseudoephedrine hydrochloride; gastrointestinal drugs such as cimetidine, loperamide hydrochloride and ranitidine; and respiratory drugs such as albuterol sulfate, aminophylline and theophylline. In certain embodiments, pharmaceutically active agents include NSAIDs such as ibuprofen, ketoprofen, carprofen, fenoprofen, and naproxen. “Pharmaceutically active agents” within the scope of this invention also include nutraceuticals, vitamins, minerals, and dietary supplements. It is also envisioned that additional areas of application for the methods and particles of the invention include food preparation and personal care products (such as cosmetics).

A flavorant according to the invention is any substance that is perceived by a majority of a target group of humans as having a pleasant flavor or at least non-objectionable flavor. The flavorant can exist as a solid, oil, or aqueous liquid. Representative, non-limiting examples of flavorants include those that impart one or more of the following flavors: lemon, orange, mixed berry, cherry, strawberry, grape, cream, vanilla, chocolate, mocha, and mint.

Preferred cellulosic materials according to the invention include microcrystalline cellulose, microcrystalline cellulose coprocessed with a hydrocolloid, and any combination thereof, individually or in admixture with a hydrocolloid. Such materials are well known to those skilled in the art and include microcrystalline cellulose per se, a product sold, for example, under the designation AVICEL® PH-101 by FMC Corporation, Philadelphia, Pa. Microcrystalline cellulose also can be present as a coprocessed aggregate with a hydrocolloid in which the weight ratio of microcrystalline cellulose to hydrocolloid is from 99:1 to about 70:30 and more preferably 97.5:2.5 to about 85:15. Suitable hydrocolloids for a co-processed aggregate with microcrystalline cellulose include methylcellulose, hydroxypropylmethylcellulose, hydroxypropylcellulose, hydroxy-ethylcellulose, carboxymethylcellulose sodium, polyvinylpyrollidone, guar gum, locust bean gum, konjac, xanthan, alginates, carrageenan and combinations thereof. Certain of these cellulosic materials are disclosed as taste masking agents in U.S. Pat. No. 5,904,937, issued on May 18, 1999, in the name of Augello, et al.

In embodiments wherein the hydrocolloid is methylcellulose, the weight ratio of microcrystalline cellulose to hydrocolloid preferably is from 99:1 to about 70:30 and more preferably 97.5:2.5 to about 85:15. Coprocessed aggregates of microcrystalline cellulose and methylcellulose are prepared generally in accordance with U.S. Pat. No. 5,725,886, issued on Mar. 10, 1998, in the name of Erkoboni, et al., as a spheronizing agent useful for the production of more uniform spheres having high drug loading. Uniformly sized drug loaded spheres are disclosed as useful as a substrate for coating and inclusion in controlled release and/or sustained release drug delivery systems. Coprocessed aggregates of microcrystalline cellulose and methylcellulose are prepared in a known manner, as more fully described in the above patent. A slurry of microcrystalline cellulose in an aqueous solution of the hydrocolloid is prepared. This is accomplished by adding microcrystalline cellulose to the aqueous hydrocolloid under intense agitation such as provided by a high energy dispersator as exemplified by a Cowles brand mixer or comparable device. Mixing of the microcrystalline cellulose and the aqueous hydrocolloid is continued until the hydrocolloid and the microcrystalline cellulose crystallites become intimately associated. After the blending is complete, the slurry is dried, preferably by spray drying, to produce a dried coprocessed aggregate of microcrystalline cellulose and hydrocolloid that has significantly different properties from either of the separate components or of a simple blend of the two components. Conventional spray drying equipment and operating procedures are employed.

The microcrystalline cellulose or co-processed microcrystalline cellulose can also be used in combination with one another. These cellulosic materials can also be used in admixture with a: hydrocolloid such as methylcellulose, hydroxypropylmethylcellulose, hydroxypropylcellulose, hydroxy-ethylcellulose, carboxymethylcellulose sodium, guar gum, polyvinylpyrollidone, locust bean gum, konjac, xanthan, carrageenan, alginates, and combinations thereof. Cellulose ethers, particularly methylcellulose, represent a preferred class of hydrocolloids. The weight ratio of microcrystalline cellulose to hydrocolloid, when they are used in admixture, is typically in the range of about 70:30 to about 99:1, preferably 85:15 to 95:5.

In accordance with the present invention, wet granulates are prepared comprising a pharmaceutically active agent, a flavorant, a cellulosic material, and an effective amount of a pharmaceutically-acceptable solvent such as water, methanol, ethanol, propanol, or methylene chloride. Such granulates typically are prepared by mixing these ingredients, although they need not be added in any particular order. This is preferably accomplished by adding the solvent portion to a mixture of the active agent, flavorant, and cellulosic material with agitation or stirring to form a wet granulate in which the solvent is evenly distributed throughout. Sufficient solvent is added to the mixture to provide a wet granulate of requisite consistency, as required for further processing, such as by extrusion/spheronization or granulation pelletization in a high shear granulator, as known to one skilled in the art.

The granulates of the invention preferably are used to prepare particles such as spheres, elliptoids, cylinders and granules having relatively smooth and uniform surfaces. Particles according to the invention need not be of the same size, although they preferably will have an average diameter of up to about 1000 micrometers (as measured by sieve analysis), preferably from about 100 to about 1000 micrometers, more preferably from about 200 to about 900 micrometers. The particles of the invention can include those with surface irregularities. Preferred particles have a relatively smooth, uniform surface.

The particles of the invention can be prepared by any of the suitable techniques known in the art. The wet granulate can be extruded through a screen having openings of about 0.5 to about 2.5 mm (preferably about 0.6 to 2.0, and most preferably about 0.8 to about 1.5 mm), using an extruder such as a Nica E-140 device, to produce compacted, spaghetti-like or ribbon-like strands or extrudate. The extrudate can be rounded using a spheronizer such as a Nica S-450 device. Under the tumbling/roping-like action of the rotating disk of the spheronizer, the cylindrical strands are broken into smaller segments which undergo smoothing and rounding to form the rounded particles which are then dried. For a more detailed description of the spheronization process, reference is made to Reynolds, “A New Technique for the Production of Spherical Particles,” Manufacturing Chemist, Aerosol News 1970, 41, 40. Once formed, the particles of the invention preferably are dried at elevated temperature suitable to maintain the homogeneity of the particle and eliminate migration of any of the components. The particles should be dried to a moisture content of less than about 5%, preferably 3-5%, by any conventional drying means.

Alternatively, suitably shaped particles can be prepared by granulation pelletization by prolonged granulation of the granulate in a high shear granulator such as a PowerEx model VG-25 device, available from Glatt Air Techniques. In this embodiment of the present invention, about 40 to about 95 parts by weight of pharmaceutically active agent, about 0.01 to about 25 parts by weight of flavorant and about 1 to about 60 parts by weight of the cellulosics material are blended in a high shear granulator until mixing is complete. Then, about 10 to about 70 parts by weight of water per 100 parts by weight of dry blend is fed to the granulator by gravity feed through a spray nozzle, increasing the blade speed and continuing granulation until the resulting rounded particles have a relatively smooth uniform surface, and preferably a mean particle size in the range of about 200 to about 900 micrometers. The resulting rounded particles may then be dried at an elevated temperature or by other suitable means.

Those skilled in the art will appreciate the usefulness of the particles of the present invention in preparing pharmaceutical dosage forms such as those that are chewed rather than swallowed as a compressed tablet. Those skilled in the art will also appreciate that for certain pharmaceutically active agents properties such as pH of the particles according to the invention may need to be adjusted in order to attain the desired degree of taste reduction. The present invention also contemplates and includes the addition of other adjuvants into the particle. The compositions of the present invention including particles and tablets may additionally contain other adjunct components conventionally found in pharmaceutical compositions, at their art-established usage levels. Thus, for example, the compositions may contain additional materials useful in physically formulating various other dosage forms of the particle of present invention, such as colorants, additional flavorants, preservatives, antioxidants, opacifiers, thickening agents and stabilizers. However, such materials, when added, should not unduly interfere with the biological activities of the pharmaceutically active agents of the compositions of the invention.

Pharmaceutical dosage forms, such as tablets, containing the particles of the invention can be prepared according to conventional techniques well known in the pharmaceutical industry. Such techniques include steps as blending and compression. The particles of the present invention can be blended with excipients required to impart the requisite compression characteristics to the final blend and the desired organoleptic properties to the chewable dosage form. In general, the dosage form is prepared by uniformly blending the particles of this invention with excipients comprising binders (such as microcrystalline cellulose, lactose, sucrose, starch, maltodextrin), disintegrants (such as starch, alginic acid, croscarmellose, polyvinylpyrollidone and sodium starch glycolate), sweeteners (such as sucrose, glucose, fructose and dextrose), artificial sweeteners (such as aspartame, saccharin and acetosulfam), lubricants (such as stearic acid, hydrogenated vegetable oils, talc and metallic stearates glycerol monosterate, polyethyleneglycol), glidants (such as silica), colorants, and additional flavorants and compressing the final blended materials into a tablet. The order of addition for the blending step, blending times, compression parameters and target properties of the compressed tablet are those typically employed by persons skilled in the art.

The following is one exemplary procedure for granulation and spheronization of ibuprofen. Additional objects, advantages, and novel features of this invention will become apparent to those skilled in the art upon examination of this example, which is not intended to be limiting. The granulate was made using a co-processed microcrystalline cellulose/methyl cellulose having a 95/5 weight ratio of microcrystalline cellulose and methylcellulose (METHOCEL A-15LV) as disclosed in Example 1 of U.S. Pat. No. 5,725,866.

### EXAMPLE 1

In the bowl of a Hobart mixer were placed 850 grams of ibuprofen, 133.2 grams of the co-processed 95/5 microcrystalline cellulose/methyl cellulose, 7.5 grams of methylcellulose (METHOCEL A4M Premium; Dow Chemical, Midland Mich.), 2.33 grams of Prosweet powder (Virginia Dare), 4.22 grams of lemon flavor powder (Firmenich), and 2.8 grams of Aspartame (Nutrasweet AG). This dry mixture was blended for 5 minutes. Deionized water (410 grams) was fed in slowly while the mixer was operated at low speed. When the water had been completely added, the bowl was scraped. The bowl was scraped again 5 minutes later. Mixing was continued such that the total time for wet mix granulation was 15 minutes. The granulation mixture was extruded through a Nika E-140 extruder using a screen having 0.8 mm drilled openings, and then spheronized using a Nika S-450 spheronizer. The resulting spheres were then dried in a Blue M oven at 50EC for 14 hours. The particles made by this process were somewhat round with sizes ranging from less than 840 microns (20 mesh) to more than 250 micron (60 mesh). Sieve fractions on the 30 mesh, 40 mesh, and 45 mesh screens were retained with the oversize (>20 mesh) and undersize (<45 mesh) particles and set aside. When tasted, the lemon flavor of the particles was not apparent, and the taste and burn associated with ibuprofen were reduced.

### EXAMPLE 2

Dry ingredients were charged to an 8 qt Patterson Kelly blender. 850 grams of ibuprofen, 133.17 grams of co-processed 95/5 microcrystalline cellulose/methyl cellulose, 7.5 grams of METHOCEL A4M Premium, 2.33 grams of Prosweet powder, 4.22 grams of lemon flavor powder and 2.8 grams of Aspartame. This dry mixture was blended for 5 minutes. The dry mixture was then charged to a Hobart mixer and 390 grams of deionized water were fed in slowly while the mixer was operated at slow speed. When the water had been completely added, the bowl was scraped after 7 minutes of mixing. The bowl was scraped again 5 minutes later. Mixing was continued such that the total time for wet mix granulation was 15 minutes. The granulation mixture was extruded through a screen having 0.6 millimeter by 0.7 millimeter openings and then spheronized using a Niro extruder/spheronizer. The resulting particles were then dried in a Blue M oven at 50° C. for fourteen hours.

The particles were fractionated by sieving through a series of fine mesh screens. A lo large diameter sample (355 microns to 600 microns) was prepared by sieving the unfractionated particles through a series of 25 mesh, 30 mesh, 40 mesh and 45 mesh screens, respectively. The oversized portion (retained on the 25 mesh) and the undersize portion (which passed through the 45 mesh) were not used. An assay of large diameter sample (i.e., the mixture of the 30 mesh, 40 mesh and 45 mesh fractions) for ibuprofen was 99.5% based on the theoretical batch content of 85% ibuprofen. A small diameter sample (diameter of 150 microns to 425 microns) was prepared by sieving a second batch of unfractionated particles through a series of 35 mesh, 40 mesh, 60 mesh, 80 mesh and 100 mesh screens. The oversize portion (retained on 35 mesh) and undersize portion (which passed through 100 mesh) were not used. The small diameter sample (i.e., the mixture of the 40, 60, 80 and 100 mesh fractions) assayed for ibuprofen content was 99.5% of the theoretical ibuprofen content, the same as determined for the large diameter sample.

Chewable 100 mg ibuprofen tablets were prepared using the large and small diameter samples, respectively. Each batch was prepared as follows: 58.82 grams of particles (theoretical 85% ibuprofen), 58.86 grams of glycerol monostearate (Eastman Chemical), 50 grams of starch 1500 (Colorcon), 3 grams of Durarome lemon flavor (Firmenich) and 6 gram of Aspartame (Nutrasweet) were charged to a 2 quart laboratory powder blender and mixed for 10 minutes. The following ingredients were added to the mixture: 134.2 grams of sorbitol (Ruger), 34.55 grams of mannitol (Ruger), 5.0 grams of Ac-Di-Sol® croscarmellose sodium (FMC) and 5.0 grams of citric acid. The mixture was blended for an additional 10 minutes. Then 3.88 grams of stearic acid were added and the mixture was blended for 5 more minutes. Finally 3.87 grams of magnesium stearate were added and the mixture was blended for 3 additional minutes. (The sorbitol and mannitol were sieved through a 20 mesh screen and the aspartame was sieved using a 40 mesh screen prior to addition.) The final powder blends were tabletted using a Stokes 512 tablet press fitted with 12.5 mm round, flat-faced tooling to provide tablets with a typical weight of 746 mg and a hardness of 5 to 6 kp. The tablets prepared using the particles in small diameter sample had an ibuprofen assay of 96.5% compared to theoretical 100 mg of ibuprofen per tablet, which was attributed to a low tablet weight of 740 mg. Tablets prepared using the particles in the large diameter sample gave a low assay of 91.1% compared to theoretical 100 mg of ibuprofen per tablet, which was attributed to segregation of the larger particles during the tabletting process. The powder blend containing the large particle sample was assayed at 100.7% ibuprofen compared to theoretical batch charge. Chewable tablets containing the large and the small fractionated particles were effectively tastemasked. The ibuprofen throat burn was significantly lessened for tablets containing with the particles compared to chewable children's Motrin ibuprofen tablets of the same strength.

### EXAMPLE 3

Two additional batches of particles were prepared following the procedure of Example 2 with the following ingredients: 800 grams of ibuprofen, 142.5 grams of coprocessed 95/5 microcrystalline cellulose/methyl cellulose, 7.5 grams of hydroxypropyl cellulose (METHOCEL E4M, Dow Chemical) and 50 grams of flavor powder.

One batch of particles was prepared that contained a vanilla cream flavor powder (Firmenich) and used 245 grams of water in the granulation step. Weight loss after drying was 0.16%. After sieve fractionation to obtain a fraction with a nominal size of 150 to 425 microns as described in Example 2, the particles were assayed at 99% of the theoretical ibuprofen content of 80%.

Another batch of particles was prepared that contained a lemon flavor powder (Firmenich) and used 250 grams of water during the granulation step. Weight loss after drying for 14 hours was 0.08%. After sieve fractionation to give a nominal size of 150 to 425 microns, the particles were assayed at 100% of the theoretical ibuprofen content of 80%.

Chewable tablets containing a nominal 100 mg of ibuprofen were prepared using the lemon and vanilla cream particles, respectively. Each batch of tablets was prepared as in Example 2 using a powder blend of the following ingredients: 62.54 grams of ibuprofen particles (80%), 55.18 grams of glycerol monostearate (Eastman Chemical), 50 grams of starch 1500 (Colorcon), 3 grams of Durarome lemon flavor (Firmenich) and 6 gram of aspartame (Nutrasweet), 134.2 grams of sorbitol (Ruger), 34.55 grams of mannitol (Ruger), 5.0 grams of Ac-Di-Sol® croscarmellose sodium (FMC), 5.0 grams of citric acid, 3.88 grams of stearic acid, and 3.87 grams of magnesium stearate. The powder blend with the particles containing the vanilla cream flavor had an assay of 99.25% and the tablets had an assay of 101.5% compared to theoretical. The powder blend with the lemon-containing particles had an assay of 103.7% and the tablets prepared from this blend had an assay of 102.7% theoretical. When tasted, the chewable tablets containing the lemon and the vanilla cream particles provided equivalent tastemasking performance and were indistinguishable for throat burn. When tasted by several individuals, some had a personal preference for the stronger lemon flavor provided by the chewable tablet containing the lemon particles compared to the smoother lemon taste observed for the chewable tablets which contained the vanilla particles.

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

What is claimed is:

1. A method comprising the step of forming particles from a wet granulate that includes a solvent portion and a non-solvent portion, wherein said solvent portion includes a pharmaceutically-acceptable solvent and said non-solvent portion comprises:
at least one pharmaceutically active agent;
at least one flavorant; and
at least one cellulosic material that is microcrystalline cellulose, microcrystalline cellulose coprocessed with a hydrocolloid, or any combination thereof, individually or in admixture with a hydrocolloid.

2. The method of claim 1 wherein said pharmaceutically active agent constitutes about 40 to about 95 weight percent of said non-solvent portion, said flavorant constitutes about 0.01 to about 25 weight percent of said non-solvent portion, and said cellulosic material constitutes about 1 to about 60 weight percent of said non-solvent portion.

3. The method of claim 1 wherein said pharmaceutically active agent constitutes about 60 to about 95 weight percent of said non-solvent portion, said flavorant constitutes about 0.01 to about 15 weight percent of said non-solvent portion, and said cellulosic material constitutes about 1 to about 40 weight percent of said non-solvent portion.

4. The method of claim 1 wherein said pharmaceutically active agent constitutes about 70 to about 95 weight percent of said non-solvent portion, said flavorant constitutes about 0.01 to about 10 weight percent of said non-solvent portion, and said cellulosic material constitutes about 1 to about 30 weight percent of said non-solvent portion.

5. The method of claim 1 wherein said pharmaceutically active agent is ibuprofen.

6. The method of claim 1 wherein said hydrocolloid is a cellulose ether.

7. The method of claim 1 wherein said particles are formed by extruding said granulate through a screen and processing said resulting extrudate using a spheronizer.

8. The method of claim 1 wherein said particles are formed by pelletizing said granulate using a high shear granulator.

9. The method of claim 1 further comprising dry blending said pharmaceutically active agent, said flavorant, and said cellulosic material to form a dry blend.

10. The method of claim 9 further comprising mixing solvent with said pharmaceutically active agent, said flavorant, and said cellulosic material for a time and under conditions effective to prepare said granulate.

11. The method of claim 1 further comprising compressing a composition comprising a plurality of said particles into a tablet.

12. A particle prepared by the method of claim 1.

13. A tablet prepared by the method of claim 11.

14. A method comprising the step of compressing a composition comprising a plurality of particles into a tablet, wherein said particles individually include:
at least one pharmaceutically active agent;
at least one flavorant; and
at least one cellulosic material that is microcrystalline cellulose, microcrystalline cellulose coprocessed with a hydrocolloid, or any combination thereof, individually or in admixture with a hydrocolloid.

15. The method of claim 14 wherein said pharmaceutically active agent constitutes about 40 to about 95 weight percent of said particles, said flavorant constitutes about 0.01 to about 25 weight percent of said particles, and said cellulosic material constitutes about 1 to about 60 weight percent of said particles.

16. The method of claim 14 wherein said pharmaceutically active agent constitutes about 60 to about 95 weight percent of said particles, said flavorant constitutes about 0.01 to about 15 weight percent of said particles, and said cellulosic material constitutes about 1 to about 40 weight percent of said particles.

17. The method of claim 14 wherein said pharmaceutically active agent constitutes about 70 to about 95 weight percent of said particles, said flavorant constitutes about 0.01 to about 10 weight percent of said particles, and said cellulosic material constitutes about 1 to about 30 weight percent of said particles.

18. The method of claim 14 wherein said pharmaceutically active agent is ibuprofen.

19. A tablet prepared by the method of claim 14.

20. A particle that comprises:
at least one pharmaceutically active agent;
at least one flavorant; and
at least one cellulosic material that is microcrystalline cellulose, microcrystalline cellulose coprocessed with a hydrocolloid, or any combination thereof, individually or in admixture with a hydrocolloid.

21. The particle of claim 20 wherein said pharmaceutically active agent is ibuprofen.

22. The particle of claim 20 wherein said hydrocolloid is a cellulose ether.

23. The particle of claim 20 that has a diameter of up to about 1000 micrometers.

24. A tablet comprising a plurality of particles that individually include:
at least one pharmaceutically active agent;
at least one flavorant; and
at least one cellulosic material that is microcrystalline cellulose, microcrystalline cellulose coprocessed with a hydrocolloid, or any combination thereof, individually or in admixture with a hydrocolloid.

25. The tablet of claim 24 wherein said pharmaceutically active agent is ibuprofen.

26. The tablet of claim 24 wherein said hydrocolloid is a cellulose ether.

27. The tablet of claim 26 wherein said cellulose ether is methyl cellulose.

28. The tablet of claim 24 further comprising at least one pharmaceutically acceptable excipient or adjuvant.

29. The method of claim 6 wherein said a cellulose ether is methyl cellulose.

30. The particle of claim 28 wherein said cellulose ether is methyl cellulose.
